(19) United States
(12) Patent Application Publication
(10) Pub. No.: US 2021/0220053 A1
(43) Pub. Date: Jul. 22, 2021

(54) LITHOPLASTY DEVICE WITH ADVANCING ENERGY WAVEFRONT

(71) Applicant: Bolt Medical, Inc., Carlsbad, CA (US)

(72) Inventor: Christopher A. Cook, Laguna Niguel, CA (US)

(21) Appl. No.: 17/149,160

(22) Filed: Jan. 14, 2021

Related U.S. Application Data
(60) Provisional application No. 62/964,529, filed on Jan. 22, 2020.

(51) Int. Cl. A61B 18/26

(52) U.S. Cl. CPC A61B 18/26; A61B 2017/00194

(57) ABSTRACT

The present invention is directed toward a method for treating a vascular lesion within or adjacent to a vessel wall. The method includes the steps of generating energy with an energy source; receiving the energy with a plurality of energy guides; and controlling the energy source with a system controller of a catheter system so that the energy from the energy source is sequentially directed to each of the plurality of energy guides in a first firing sequence. The method can include the system controller controlling a firing rate of the energy source to each of the plurality of energy guides. The method can include the system controller controlling a firing sequence to the plurality of energy guides so that an advancing wavefront is generated toward the vascular lesion from near a balloon proximal end and/or from near a balloon distal end. The system controller can control a firing energy level, which can be dependent at least partially upon the pulse width, the wavelength and/or the amplitude of the energy pulses.

### RELATED APPLICATION

This application claims priority on U.S. Provisional Patent Application Ser. No. 62/964,529, filed on Jan. 21, 2020, entitled “LITHOPLASTY DEVICE WITH ADVANCING ENERGY WAVEFRONT”. As far as permitted, the contents of U.S. Provisional Application Ser. No. 62/964,529 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

### SUMMARY

The present invention is directed toward a method for treating a vascular lesion within or adjacent to a vessel wall. In various embodiments, the method includes the steps of generating energy with an energy source; receiving the energy with a plurality of energy guides; and controlling the energy source with a system controller of a catheter system so that the energy from the energy source is sequentially directed to each of the plurality of energy guides in a first firing sequence.

In some embodiments, the step of controlling can include the system controller controlling a firing rate of the energy source to each of the plurality of energy guides.

In certain embodiments, the step of controlling can include the system controller controlling the energy source so that the energy from the energy source is alternatively directed to each of the plurality of energy guides at a first firing rate and a second firing rate that is different than the first firing rate.

In various embodiments, the step of receiving can include the plurality of energy guides including a first energy guide and a second energy guide. The method can also include the steps of positioning a first guide distal end of the first energy guide at a first longitudinal position along a length of the balloon, and positioning a second guide distal end of the second energy guide at a second longitudinal position along the length of the balloon so that the first longitudinal position is different than the second longitudinal position.

In some embodiments, the step of receiving can include the plurality of energy guides including a first energy guide and a second energy guide. The method can also include the steps of positioning a first guide distal end of the first energy guide at a first longitudinal position along a length of the balloon, and positioning a second guide distal end of the second energy guide at a second longitudinal position along the length of the balloon so that the first longitudinal position is the same as the second longitudinal position.

In certain embodiments, the step of positioning can include at least a portion of the energy guides being positioned within a balloon that is coupled to a catheter shaft.

In various embodiments, the step of controlling can include the system controller controlling a firing sequence to the plurality of energy guides so that an advancing wavefront is generated toward the vascular lesion from near a balloon proximal end and from near a balloon distal end.

In some embodiments, the step of controlling can include the system controller controlling the energy source so that the energy from the energy source is alternatively directed to at least two of the plurality of energy guides at a different firing rate from one another.

In certain embodiments, the step of controlling can include the system controller controlling the energy source so that the energy from the energy source is alternatively directed to at least two of the plurality of energy guides at a different firing energy level from one another.

In various embodiments, the firing energy level can be dependent at least partially upon the pulse width of the energy pulses.

In some embodiments, the firing energy level can be dependent at least partially upon the wavelength of the energy pulses.

In certain embodiments, the firing energy level can be dependent at least partially upon the amplitude of the energy pulses.

In various embodiments, the step of controlling can include the system controller controlling a firing sequence to the plurality of energy guides so that an advancing wavefront is generated toward the vascular lesion in a direction from one of a balloon proximal end and a balloon distal end.

In some embodiments, the step of controlling can include the system controller controlling the energy source so that the energy from the energy source is alternatively directed to at least two of the plurality of energy guides at a different firing rate from one another.

In various embodiments, the step of controlling can include the system controller controlling the energy source so that the energy from the energy source is alternatively directed to at least two of the plurality of energy guides at a different firing energy level from one another.

In various embodiments, each of the plurality of energy guides can include an optical fiber.

In some embodiments, the energy source can be a laser source that generates laser energy.

In certain embodiments, the energy source can be an energy source that generates electrical impulses.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of an embodiment of a catheter system in accordance with various embodiments herein, the catheter system including a plurality of energy guides and a multiplexer;

FIG. 2A is a simplified schematic top view of a portion of an embodiment of the catheter system including one embodiment of the multiplexer;

FIG. 2B is a simplified schematic perspective view of a portion of the catheter system and the multiplexer illustrated in FIG. 2A;

FIG. 3A is a simplified schematic top view of a portion of an embodiment of the catheter system including another embodiment of the multiplexer;

FIG. 3B is a simplified schematic perspective view of a portion of the catheter system and the multiplexer illustrated in FIG. 3A;

FIG. 4 is a simplified schematic top view of a portion of the catheter system and still another embodiment of the multiplexer;

FIG. 5 is a simplified schematic top view of a portion of the catheter system and yet another embodiment of the multiplexer;

FIG. 6 is a simplified schematic top view of a portion of the catheter system and another embodiment of the multiplexer;

FIG. 7 is a simplified schematic top view of a portion of the catheter system and still another embodiment of the multiplexer;

FIG. 8A is a simplified schematic side view of a portion of another embodiment of the catheter system;

FIG. 8B is a simplified schematic cross-sectional view of the portion of the catheter system taken on line B-B in FIG. 8A;

FIG. 9 is a simplified schematic cross-sectional view of another embodiment of the catheter system;

FIG. 10 is a simplified schematic cross-sectional view of still another embodiment of the catheter system;

FIG. 11 is a simplified schematic cross-sectional view of yet another embodiment of the catheter system;

FIG. 12 is a simplified schematic cross-sectional view of another embodiment of the catheter system; and

FIG. 13 is a simplified schematic cross-sectional view of still another embodiment of the catheter system.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In a pressure wave generating medical device, it is often desirable to have a number of potential output channels for the treatment process. For safety and convenience, these output channels can consist of optical fibers. Since a high-power laser source is often the largest and most expensive component in the system, it can be advantageous to utilize a single laser source that can be multiplexed into a number of different optical fibers for treatment purposes. This allows the possibility for using all of the laser power with each optical fiber. However, although the present invention is often described herein as using a single laser source for purposes of generating the desired pressure waves, it is appreciated that the present invention is not limited to the use of a laser-generated pressure wave system. For example, the present invention can alternatively use any suitable type of device that utilizes a highly localized energy source to generate the desired pressure waves. In one non-exclusive alternative example, the energy source can generate electrical impulses that are directed through the energy guides to generate the desired pressure waves. It is appreciated that the present invention can also utilize more than one laser source and/or more than one other suitable pressure wave generating device.

Thus, in various embodiments, the catheter systems and related methods disclosed herein are configured to provide a means to power multiple fiber optic channels in a pressure wave generating device that is designed to impart pressure onto and induce fractures in vascular lesions, such as calcified vascular lesions and/or fibrous vascular lesions using a single energy source. As described in detail herein, in certain embodiments, the catheter systems can be configured and controlled to selectively and/or separately power the multiple fiber optic channels in any desired firing sequence, pattern, order, firing rate and/or firing duration, etc. Thus, the invention described in detail herein can include a single energy source, which can be multiplexed into one or more of a plurality of energy guides in a single use device. This allows a single, stable energy source to be channeled sequentially in any desired firing sequence and at any firing rate and/or duration through any or all of the plurality of energy guides.

It is appreciated that although each of the plurality of energy guides can be powered separately in any desired firing sequence, pattern, order, firing rate, firing duration, sets and/or subsets of the plurality of energy guides can also be powered at any given point in time. Each set or subset of the plurality of energy guides can include one or more of the plurality of energy guides. Thus, at any given point in time, power can be directed to one or more of the plurality of energy guides to alternatively create a first firing sequence, a second firing sequence, a third firing sequence, a fourth firing sequence, etc. Moreover, in various applications of the present invention, each firing sequence of the energy guides in such sets and subsets of the plurality of energy guides can be different than one or more of the other firing sequences of the energy guides.

As provided herein, the catheter systems can utilize light energy from the energy source, i.e. a laser source or other suitable energy source, to generate a plasma within the balloon fluid at or near a guide distal end of each of the plurality of energy guides disposed in the balloon located at a treatment site. The plasma formation can initiate one or more pressure waves by initiating rapid formation of one or more bubbles that can rapidly expand to a maximum size and then dissipate through a cavitation event that can launch a pressure wave upon collapse. Alternatively, the plasma formation can initiate an explosive type of pressure wave or pressure waves that can extend to the treatment site to disrupt calcification. Stated another way, the rapid expansion of the plasma-induced bubbles can generate one or more pressure waves within the balloon fluid retained within the balloon and thereby impart pressure waves upon the treatment site.

As provided herein, a guide distal end of each of the plurality of energy guides can be positioned in any suitable locations relative to a length of the balloon to more effectively and precisely impart pressure waves for purposes of disrupting the vascular lesions at the treatment site. Further, as noted, with the configuration of the present invention, it is possible to fire individual energy guides, including one or more energy guides that are fired substantially simultaneously or sequentially, to achieve a firing sequence or pattern that could be more effective at disrupting localized lesions. Firing separate plasma generator channels in a predetermined and/or specific firing sequence or pattern can create a moving energy wavefront that more effectively breaks up a lesion in one location or an extended lesion.

As used herein, the terms “intravascular lesion”, “vascular lesion” and “treatment site” are used interchangeably unless otherwise noted, and can also include lesions located at or near heart valves. The intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

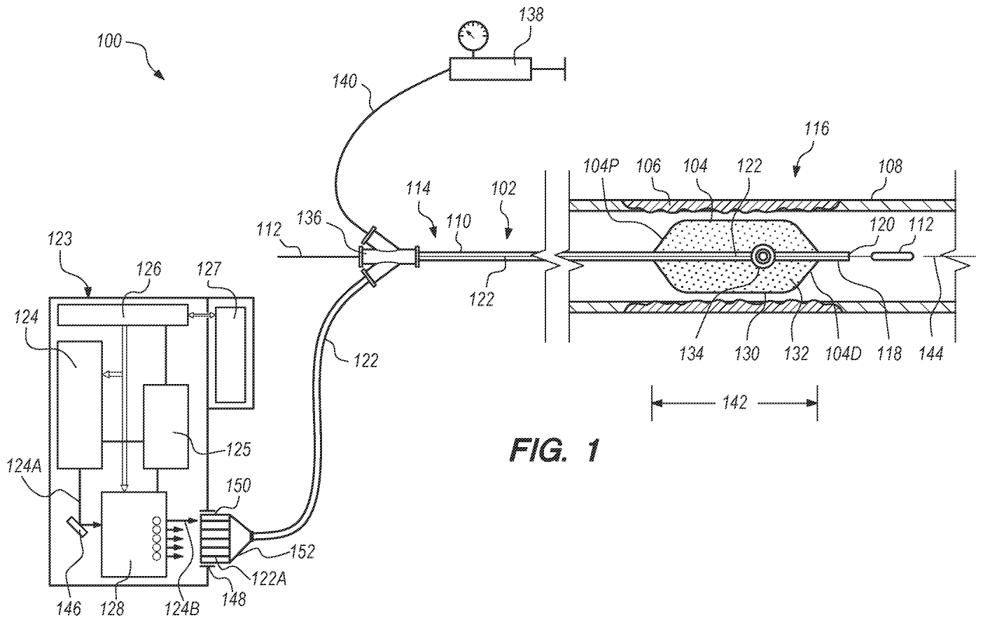

It is appreciated that the catheter systems disclosed herein can include many different forms. Referring now to FIG. 1, a schematic cross-sectional view is shown of a catheter system 100 in accordance with various embodiments herein. As described herein, the catheter system 100 is suitable for imparting pressure to induce fractures in one or more vascular lesions within or adjacent a vessel wall of a blood vessel or heart valve. In the embodiment illustrated in FIG. 1, the catheter system 100 can include one or more of a catheter 102, an energy guide bundle 122 including a plurality of energy guides 122A, a source manifold 136, a fluid pump 138, and a system console 123 including one or more of an energy source 124, a power source 125, a system controller 126, a graphic user interface 127 (a “GUI”), and a multiplexer 128.

The catheter 102 is configured to move to a treatment site 106 within or adjacent to a blood vessel 108. The treatment site 106 can include one or more vascular lesions such as calcified vascular lesions, for example. Additionally, or in the alternative, the treatment site 106 can include vascular lesions such as fibrous vascular lesions.

The catheter 102 can include an inflatable balloon 104 (sometimes referred to herein simply as a “balloon”), a catheter shaft 110 and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter shaft 110 can also include a guidewire lumen 118 which is configured to move over the guidewire 112. The catheter shaft 110 can further include an inflation lumen (not shown). In some embodiments, the catheter 102 can have a distal end opening 120 and can accommodate and be tracked over the guidewire 112 as the catheter 102 is moved and positioned at or near the treatment site 106.

In various embodiments, the catheter shaft 110 of the catheter 102 can be coupled to the plurality of energy guides 122A of the energy guide bundle 122 that can be in optical and/or electrical communication with the energy source 124. The energy guide(s) 122A can be disposed along the catheter shaft 110 and within the balloon 104. Additionally, each of the energy guides 122A can have a guide distal end (not shown in FIG. 1) that is at any suitable longitudinal position relative to a length 142 of the balloon 104 and/or relative to a length of the guidewire lumen 118. In some embodiments, each energy guide 122A can be an optical fiber and the energy source 124 can be a laser. The energy source 124 can be in optical and/or electrical communication with the energy guides 122A at the proximal portion 114 of the catheter system 100. More particularly, as described in detail herein, the energy source 124 can selectively and/or alternatively be in optical and/or electrical communication with each of the energy guides 122A due to the presence and operation of the multiplexer 128. Alternatively, each energy guide 122A can have another suitable design and/or the energy source 124 can be another suitable energy source.

In some embodiments, the catheter shaft 110 can be coupled to multiple energy guides such as a first energy guide, a second energy guide, a third energy guide, etc., which can be disposed at any suitable positions about the guidewire lumen 118 and/or the catheter shaft 110. For example, in certain non-exclusive embodiments, two energy guides 122A can be spaced apart by approximately 180 degrees about the circumference of the guidewire lumen 118 and/or the catheter shaft 110; three energy guides 122A can be spaced apart by approximately 120 degrees about the circumference of the guidewire lumen 118 and/or the catheter shaft 110; four energy guides 122A can be spaced apart by approximately 90 degrees about the circumference of the guidewire lumen 118 and/or the catheter shaft 110; five energy guides 122A can be spaced apart by approximately 72 degrees about the circumference of the guidewire lumen 118 and/or the catheter shaft 110; six energy guides 122A can be spaced apart by approximately 60 degrees about the circumference of the guidewire lumen 118 and/or the catheter shaft 110; or eight energy guides 122A can be spaced apart by approximately 45 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A described herein can be disposed uniformly or non-uniformly about the guidewire lumen 118 and/or the catheter shaft 110 to achieve the desired effect in the desired locations.

The balloon 104 can include a balloon wall 130 and can be inflated with a balloon fluid 132 to expand from a collapsed configuration suitable for advancing the catheter 102 through a patient's vasculature, to an expanded configuration suitable for anchoring the catheter 102 in position relative to the treatment site 106. Stated in another manner, when the balloon 104 is in the expanded configuration, the balloon wall 130 of the balloon 104 is configured to be positioned substantially adjacent to the treatment site 106. In some embodiments, the energy source 124 of the catheter system 100 can be configured to provide sub-millisecond pulses of light from the energy source 124, along the energy guides 122A, to a location within the balloon 104, thereby inducing plasma formation in the balloon fluid 132 within the balloon 104. Although not intending to be bound by any one particular theory, it is believed that the plasma formation causes rapid bubble formation, and imparts pressure waves upon the treatment site 106, although other mechanisms of imparting pressure waves are contemplated. Exemplary plasma-induced bubbles are shown as bubbles 134 in FIG. 1. The balloon fluid 132 can be a liquid or a gas.

The balloons 104 suitable for use in the catheter systems 100 described in detail herein include those that can be passed through the vasculature of a patient when in the collapsed configuration. In some embodiments, the balloons 104 herein are made from silicone. In other embodiments, the balloons 104 herein are made from polydimethylsiloxane (PDMS), polyurethane, polymers such as PEBAX™ material available from Arkema, which has a location at King of Prussia, Pa., USA, nylon, and the like. In some embodiments, the balloons 104 can include those having diameters ranging from one millimeter (mm) to 25 mm in diameter. In some embodiments, the balloons 104 can include those having diameters ranging from at least 1.5 mm to 12 mm in diameter. In some embodiments, the balloons 104 can include those having diameters ranging from at least one mm to five mm in diameter.

Additionally, in some embodiments, the balloons 104 herein can include a length 142 ranging from at least approximately five mm to 300 mm. More particularly, in some embodiments, the balloons 104 herein can include those having a length 142 ranging from at least approximately eight mm to 200 mm. It is appreciated that balloons 104 of greater length can be positioned adjacent to relatively large treatment sites 106, and, thus, may be usable for imparting pressure onto and inducing fractures in larger vascular lesions or multiple vascular lesions at specific locations within the treatment site 106.

Further, the balloons 104 herein can be inflated to inflation pressures of between approximately one atmosphere (atm) to 70 atm. In some embodiments, the balloons 104 herein can be inflated to inflation pressures of from at least approximately 20 atm to 70 atm. In other embodiments, the balloons 104 herein can be inflated to inflation pressures of from at least approximately six atm to 20 atm. In still other embodiments, the balloons 104 herein can be inflated to inflation pressures of from at least approximately three atm to 20 atm. In yet other embodiments, the balloons 104 herein can be inflated to inflation pressures of from at least approximately two atm to ten atm.

The balloons 104 herein can have various shapes including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloons 104 herein can include a drug eluting coating or a drug eluting stent structure. The drug eluting coating or drug eluting stent can include one or more therapeutic agents including anti-inflammatory agents, anti-neoplastic agents, anti-angiogenic agents, and the like.

Exemplary balloon fluids 132 suitable for use herein can include, but are not limited to one or more of water, saline, contrast medium, fluorocarbons, perfluorocarbons, gases, such as carbon dioxide, and the like. In some embodiments, the balloon fluids 132 described can be used as base inflation fluids. In some embodiments, the balloon fluids 132 include a mixture of saline to contrast medium in a volume ratio of 50:50. In other embodiments, the balloon fluids 132 include a mixture of saline to contrast medium in a volume ratio of 25:75. In still other embodiments, the balloon fluids 132 include a mixture of saline to contrast medium in a volume ratio of 75:25. Additionally, the balloon fluids 132 suitable for use herein can be tailored on the basis of composition, viscosity, and the like in order to manipulate the rate of travel of the pressure waves therein. In certain embodiments, the balloon fluids 132 suitable for use herein are biocompatible. A volume of balloon fluid 132 can be tailored by the chosen energy source 124 and the type of balloon fluid 132 used.

In some embodiments, the contrast agents used in the contrast media herein can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

Additionally, the balloon fluids 132 herein can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the balloon fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system. By way of non-limiting examples, various lasers described herein can include neodymium:yttrium-aluminum-garnet (Nd:YAG—emission maximum=1064 nm) lasers, holmium:YAG (Ho:YAG—emission maximum=2.1 μm) lasers, or erbium:YAG (Er:YAG—emission maximum=2.94 μm). In some embodiments, the absorptive agents used herein can be water soluble. In other embodiments, the absorptive agents used herein are not water soluble. In some embodiments, the absorptive agents used in the balloon fluids 132 herein can be tailored to match the peak emission of the energy source 124. Various energy sources 124 having emission wavelengths of at least ten nanometers to one millimeter are discussed elsewhere herein.

It is appreciated that the catheter system 100 and/or the energy guide bundle 122 disclosed herein can include any number of energy guides 122A in optical communication with the energy source 124 at the proximal portion 114, and with the balloon fluid 132 within the balloon 104 at the distal portion 116. For example, in some embodiments, the catheter system 100 and/or the energy guide bundle 122 can include from one energy guide 122A to five energy guides 122A. In other embodiments, the catheter system 100 and/or the energy guide bundle 122 can include from five energy guides 122A to fifteen energy guides 122A. In yet other embodiments, the catheter system 100 and/or the energy guide bundle 122 can include from ten energy guides 122A to 30 energy guides 122A. Alternatively, in still other embodiments, the catheter system 100 and/or the energy guide bundle 122 can include greater than 30 energy guides 122A.

Additionally, it is further appreciated that the energy guides 122A can be disposed at any suitable positions about the circumference of the guidewire lumen 118 and/or the catheter shaft 110, and the guide distal end of each of the energy guides 122A can be disposed at any suitable longitudinal position relative to the length 142 of the balloon 104 and/or relative to the length of the guidewire lumen 118. Moreover, it is also appreciated that at least a portion of one or more of the energy guides 122A can be positioned spaced apart from the guidewire lumen 118, e.g., the guide distal end of such energy guides 122A can be positioned at any suitable position laterally between the guidewire lumen 118 and the balloon wall 130 of the balloon 104.

Further, the energy guides 122A herein can assume many configurations about and/or relative to the catheter shaft 110 of the catheters 102 described herein. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A herein can be disposed within one or more energy guide lumens (not shown) within the catheter shaft 110.

In various embodiments, the energy guides 122A herein can each include an optical fiber or flexible light pipe. The energy guides 122A herein can be thin and flexible and can allow light signals to be sent with very little loss of strength. The energy guides 122A herein can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide light along its length to a distal portion, i.e. the guide distal end, which can have one or more optical windows. The energy guides 122A can create a light path as a portion of an optical network including the energy source 124. The light path within the optical network allows light to travel from one part of the network to another. Both the optical fiber and the flexible light pipe can provide a light path within the optical networks herein.

Further, the energy guides 122A herein can include one or more photoacoustic transducers (not shown), where each photoacoustic transducer can be in optical communication with the energy guide 122A within which it is disposed. In some embodiments, the photoacoustic transducers can be in optical communication with the guide distal end of the energy guide 122A. Additionally, in such embodiments, the photoacoustic transducers can have a shape that corresponds with and/or conforms to the guide distal end of the energy guide 122A.

The photoacoustic transducer is configured to convert light energy into an acoustic wave at or near the guide distal end of the energy guide 122A. It is appreciated that the direction of the acoustic wave can be tailored by changing an angle of the guide distal end of the energy guide 122A.

Additionally, it is appreciated that any or all photoacoustic transducers that may be disposed at the guide distal end of the energy guide 122A herein can assume the same shape as the guide distal end of the energy guide 122A. For example, in certain non-exclusive embodiments, the photoacoustic transducer and/or the guide distal end can have a conical shape, a convex shape, a concave shape, a bulbous shape, a square shape, a stepped shape, a half-circle shape, an ovoid shape, and the like. It is also appreciated that the energy guide 122A can further include additional photoacoustic transducers disposed along one or more side surfaces of the length of the energy guide 122A.

The energy guides 122A described herein can further include one or more diverting features (not shown) or “diverters” within the energy guide 122A that are configured to direct light to exit the energy guide 122A toward a side surface e.g., at or near the guide distal end of the energy guide 122A, and toward the balloon wall 130. A diverting feature can include any feature of the system herein that diverts light from the energy guide 122A away from its axial path toward a side surface of the energy guide 122A. Additionally, the energy guides 122A can each include one or more light windows (not shown) disposed along the longitudinal or axial surfaces of each energy guide 122A and in optical communication with a diverting feature. Stated in another manner, the diverting features herein can be configured to direct light in the energy guide 122A toward a side surface, e.g., at or near the guide distal end, where the side surface is in optical communication with a light window. The light windows can include a portion of the energy guide 122A that allows light to exit the energy guide 122A from within the energy guide 122A, such as a portion of the energy guide 122A lacking a cladding material on or about the energy guide 122A.

Examples of the diverting features suitable for use herein include a reflecting element, a refracting element, and a fiber diffuser. Additionally, the diverting features suitable for focusing light away from the tip of the energy guides 122A herein can include, but are not to be limited to, those having a convex surface, a gradient-index (GRIN) lens, and a mirror focus lens. Upon contact with the diverting feature, the light is diverted within the energy guide 122A to the photoacoustic transducer that is in optical communication with a side surface of the energy guide 122A. As noted, the photoacoustic transducer then converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the plurality of energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the balloon fluid 132 as needed.

As provided herein, the system console 123 includes one or more of the energy source 124, the power source 125, the system controller 126, the GUI 127, and the multiplexer 128. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, the GUI 127 and the multiplexer 128 can be provided within the catheter system 100 without the specific need for the system console 123.

Additionally, as shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (also sometimes referred to generally as a “ferrule”) that houses a portion, e.g., a guide proximal end, of each of the energy guides 122A. The guide coupling housing 150 is configured to fit and be selectively retained within the console connection aperture 148 to provide the desired mechanical coupling between the energy guide bundle 122 and the system console 123.

Further, the energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 into the blood vessel 108 during use of the catheter system 100.

As provided herein, the energy source 124 can be selectively and/or alternatively coupled in optical communication with each of the energy guides 122A in the energy guide bundle 122. In particular, the energy source 124 is configured to generate light energy in the form of a source beam 124A, e.g., a pulsed source beam, that can be selectively and/or alternatively directed to and received by each of the energy guides 122A in the energy guide bundle 122. More specifically, as described in greater detail herein below, the source beam 124A from the energy source 124 is directed through the multiplexer 128 such that individual guide beams 124B (or “multiplexed beams”) can be selectively and/or alternatively directed into and received by each of the energy guides 122A in the energy guide bundle 122. In particular, each pulse of the energy source 124, i.e. each pulse of the source beam 124A, can be directed through the multiplexer 128 to generate a separate guide beam 124B that is selectively and/or alternatively directed onto one of the energy guides 122A in the energy guide bundle 122.

The energy source 124 can have any suitable design. In certain embodiments, as noted above, the energy source 124 can be configured to provide sub-millisecond pulses of light from the energy source 124, that are directed along the energy guides 122A, to a location within the balloon 104, thereby inducing plasma formation in the balloon fluid 132 within the balloon 104. The plasma formation causes rapid bubble formation, and imparts pressure waves upon the treatment site 106. In such embodiments, the sub-millisecond pulses of light from the energy source 124 can be delivered to the treatment site 106 at a frequency of between approximately one hertz (Hz) and 5000 Hz. In some embodiments, the sub-millisecond pulses of light from the energy source 124 can be delivered to the treatment site 106 at a frequency of between approximately 30 Hz and 1000 Hz. In other embodiments, the sub-millisecond pulses of light from the energy source 124 can be delivered to the treatment site 106 at a frequency of between approximately ten Hz and 100 Hz. In yet other embodiments, the sub-millisecond pulses of light from the energy source 124 can be delivered to the treatment site 106 at a frequency of between approximately one Hz and 30 Hz. Alternatively, the sub-millisecond pulses of light can be delivered to the treatment site 106 at a frequency that can be greater than 5000 Hz.

It is appreciated that although the energy source 124 is typically utilized to provide pulses of light energy, the energy source 124 can still be described as providing a single source beam 124A, i.e. a single pulsed source beam.

In various embodiments, the energy source 124 suitable for use herein can include various types of energy sources including, but not limited to, lasers and lamps. Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser pulse wavelengths, pulse widths and amplitudes that provide varying energy levels that can be employed to achieve plasma in the balloon fluid 132 of the catheters 102 described herein. In various embodiments, the pulse widths can include those falling within a range including from at least ten ns to 200 ns. In some embodiments, the pulse widths can include those falling within a range including from at least 20 ns to 100 ns. In other embodiments, the pulse widths can include those falling within a range including from at least one ns to 500 ns.

Additionally, exemplary nanosecond lasers can include those within the UV to IR spectrum, spanning pulse wavelengths of about ten nanometers (nm) to one millimeter (mm). In some embodiments, the energy sources 124 suitable for use in the catheter systems 100 herein can include those capable of producing light at pulse wavelengths of from at least 750 nm to 2000 nm. In other embodiments, the energy sources 124 can include those capable of producing light at pulse wavelengths of from at least 700 nm to 3000 nm. In still other embodiments, the energy sources 124 can include those capable of producing light at pulse wavelengths of from at least 100 nm to ten micrometers (μm). Nanosecond lasers can include those having repetition rates of up to 200 kHz. In some embodiments, the laser can include a Q-switched thulium:yttrium-aluminum-garnet (Tm:YAG) laser. In other embodiments, the laser can include a neodymium:yttrium-aluminum-garnet (Nd:YAG), holmium:yttrium-aluminum-garnet (Ho:YAG), erbium:yttrium-aluminum-garnet (Er:YAG), excimer laser, helium-neon laser, carbon dioxide laser, as well as doped, pulsed, fiber lasers.

The catheter systems 100 disclosed herein can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In some embodiments, the catheter systems 100 herein can generate pressure waves having maximum pressures in the range of at least two MPa to 50 MPa. In other embodiments, the catheter systems 100 herein can generate pressure waves having maximum pressures in the range of at least two MPa to 30 MPa. In yet other embodiments, the catheter systems 100 herein can generate pressure waves having maximum pressures in the range of at least 15 MPa to 25 MPa.

The pressure waves described herein can be imparted upon the treatment site 106 from a distance within a range from at least 0.1 millimeters (mm) to 25 mm extending radially from the energy guides 122A when the catheter 102 is placed at the treatment site 106. In some embodiments, the pressure waves can be imparted upon the treatment site 106 from a distance within a range from at least ten mm to 20 mm extending radially from the energy guides 122A when the catheter 102 is placed at the treatment site 106. In other embodiments, the pressure waves can be imparted upon the treatment site 106 from a distance within a range from at least one mm to ten mm extending radially from the energy guides 122A when the catheter 102 is placed at the treatment site 106. In yet other embodiments, the pressure waves can be imparted upon the treatment site 106 from a distance within a range from at least 1.5 mm to four mm extending radially from the energy guides 122A when the catheter 102 is placed at the treatment site 106. In some embodiments, the pressure waves can be imparted upon the treatment site 106 from a range of at least two MPa to 30 MPa at a distance from 0.1 mm to ten mm. In some embodiments, the pressure waves can be imparted upon the treatment site 106 from a range of at least two MPa to 25 MPa at a distance from 0.1 mm to ten mm.

The power source 125 is electrically coupled to and is configured to provide necessary power to one or more of the energy source 124, the system controller 126, and the multiplexer 128. The power source 125 can have any suitable design for such purposes.

As noted, the system controller 126 is electrically coupled to and receives power from the power source 125. Additionally, the system controller 126 is coupled to and is configured to control operation of each of the energy source 124, the GUI 127 and the multiplexer 128. The system controller 126 can include one or more processors or circuits for purposes of controlling the operation of at least the energy source 124, the GUI 127 and the multiplexer 128. For example, the system controller 126 can control the energy source 124 for generating pulses of light energy as desired, e.g., at a desired firing rate. Subsequently, the system controller 126 can then control the multiplexer 128 so that the light energy from the energy source 124, i.e. the source beam 124A, can be selectively and/or alternatively directed to each of the energy guides 122A, i.e. in the form of individual guide beams 124B, in a desired manner.

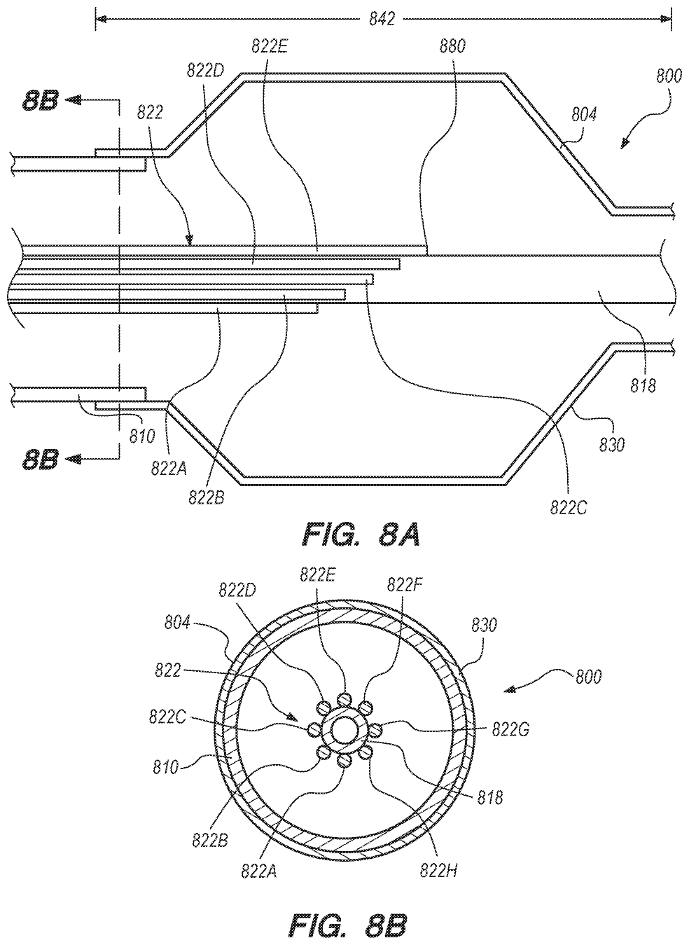

More specifically, as provided herein, the system controller 126 can control the energy source 124 and/or the multiplexer 128 so that individual guide beams 124B can be directed to each of the energy guides 122A, or sets or subsets of the energy guides 122A, in a desired firing sequence, firing pattern, firing order, firing energy levels (which can any or all of include pulse width, pulse amplitude and/or pulse wavelength) and/or firing rate. For example, in a catheter system 100 that includes eight energy guides 122A, e.g., such as shown in FIGS. 8A and 8B, that are arranged in a linear pattern with angular orientation spiraling around the guidewire lumen 118, the system controller 126 can control the sequencing of the firing of the light energy from the energy source 124 to each of the energy guides 122A in any desired manner. As used herein, the term “firing rate” is intended to mean the number of pulses per a given time frame. Further, as used herein, the term “firing energy level” is intended to mean the intensity of the energy pulse, which can be varied depending upon the pulse width and/or the pulse amplitude of any or all of the pulse(s).

Certain non-exclusive examples of alternative applications of sequencing of the firing of the eight energy guides 122A will be described in greater detail herein. As used herein, different “desired firing sequences” can equally be referred to as a first firing sequence, a second firing sequence, a third firing sequence, etc. for ease of discussion and understanding. Somewhat similarly, different firing patterns, firing orders, firing energy levels+and/or firing rates can likewise equally be referred to herein as a first, a second, a third, etc. for ease of discussion and understanding.

The system controller 126 can further be configured to control operation of other components of the catheter system 100, e.g., the positioning of the catheter 102 adjacent to the treatment site 106, the inflation of the balloon 104 with the balloon fluid 132, etc. Further, or in the alternative, the catheter system 100 can include one or more additional controllers that can be positioned in any suitable manner for purposes of controlling the various operations of the catheter system 100.

The GUI 127 is accessible by the user or operator of the catheter system 100. Additionally, the GUI 127 is electrically connected to the system controller 126. With such design, the GUI 127 can be used by the user or operator to ensure that the catheter system 100 is employed as desired to impart pressure onto and induce fractures into the vascular lesions at the treatment site 106. Additionally, the GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time, e.g., during use of the catheter system 100. Further, in various embodiments, the GUI 127 can include one or more colors, different sizes, varying brightness, etc., that may act as alerts to the user or operator. Additionally, or in the alternative, the GUI 127 can provide audio data or information to the user or operator. It is appreciated that the specifics of the GUI 127 can vary depending upon the design requirements of the catheter system 100, or the specific needs, specifications and/or desires of the user or operator.

As provided herein, where applicable, the multiplexer 128 can be configured to selectively and/or alternatively direct light energy from the energy source 124 to each of the energy guides 122A in the energy guide bundle 122. More particularly, the multiplexer 128 is configured to receive light energy from the energy source 124, e.g., a single source beam 124A from a single laser source, and selectively and/or alternatively direct such light energy in the form of individual guide beams 124B to each of the energy guides 122A in the energy guide bundle 122. As such, the multiplexer 128 enables a single energy source 124 to be channeled separately in any desired firing sequence or pattern through a plurality of energy guides 122A such that the catheter system 100 is able to impart pressure onto and induce fractures in vascular lesions at the treatment site 106 within or adjacent to a vessel wall of the blood vessel 108 in a desired manner. Additionally, as shown, the catheter system 100 can include one or more optical elements 146 for purposes of directing the light energy, e.g., the source beam 124A, from the energy source 124 to the multiplexer 128.

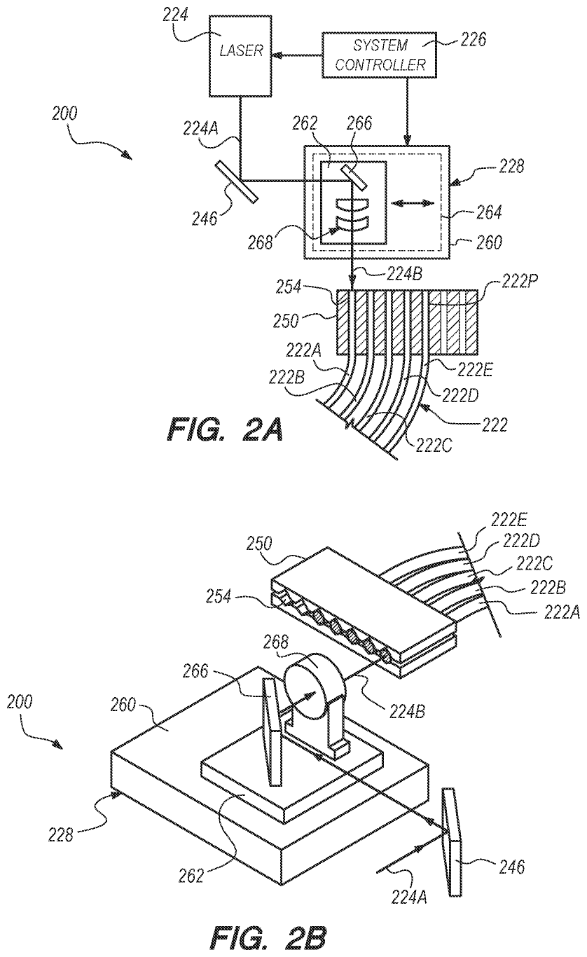

As described herein, the multiplexer 128 can have any suitable design for purposes of selectively and/or alternatively directing the light energy from the energy source 124 to each of the energy guides 122A of the energy guide bundle 122. Various non-exclusive alternative embodiments of the multiplexer 128 are described in detail herein below in relation to FIGS. 2A-7.

FIG. 2A is a simplified schematic top view of a portion of an embodiment of the catheter system 200. More particularly, FIG. 2A illustrates a plurality of energy guides, such as a first energy guide 222A, a second energy guide 222B, a third energy guide 222C, a fourth energy guide 222D and a fifth energy guide 222E, an energy source 224, a system controller 226, and an embodiment of the multiplexer 228. The multiplexer 228 receives light energy in the form of a source beam 224A, e.g., a pulsed source beam, from the energy source 224. The multiplexer 228 can selectively and/or alternatively direct the light energy in the form of individual guide beams 224B in any desired firing sequence and/or pattern (whether predetermined or otherwise) to each of the energy guides 222A-222E under control of the system controller 226. The energy guides 222A-222E, the energy source 224 and the system controller 226 are substantially similar in design and function as previously described. Accordingly, such components will not be described in detail in relation to the embodiment illustrated in FIG. 2A. It is further appreciated that certain components of the system console 123 illustrated and described above in relation to FIG. 1, e.g., the power source 125 and the GUI 127, are not illustrated in FIG. 2A for purposes of simplicity and ease of illustration, but would typically be included in many embodiments.

As noted above, the multiplexer 228 is configured to receive light energy in the form of the source beam 224A from the energy source 224 and selectively and/or alternatively direct the light energy in the form of individual guide beams 224B in any desired firing sequence and/or pattern (whether predetermined or otherwise) to each of the energy guides 222A-222E. As such, as shown in FIG. 2A, the multiplexer 228 is operatively and/or optically coupled in optical communication to the energy guide bundle 222, i.e. to the plurality of energy guides 222A-222E.

Additionally, as illustrated, a guide proximal end 222P of each of the plurality of energy guides 222A-222E is retained within a guide coupling housing 250, i.e. within guide coupling slots 254 that are formed into the guide coupling housing 250. In various embodiments, the guide coupling housing 250 is configured to be selectively coupled to the system console 123 (illustrated in FIG. 1) so that the guide coupling slots 254, and thus the energy guides 222A-222E, are maintained in a desired fixed position relative to the multiplexer 228 during use of the catheter system 200. In some embodiments, the guide coupling slots 254 are provided in the form of V-grooves, such as in a V-groove ferrule block commonly used in multichannel fiber optics communication systems. Alternatively, the guide coupling slots 254 can have another suitable design.

It is appreciated that the guide coupling housing 250 can have any suitable number of guide coupling slots 254, which can be positioned and/or oriented relative to one another in any suitable manner, e.g., to better align the guide coupling slots 254 and thus the energy guides 222A-222E relative to the multiplexer 228. In the embodiment illustrated in FIG. 2A, the guide coupling housing 250 includes seven guide coupling slots 254 that are spaced apart in a linear arrangement relative to one another, with precise interval spacing between adjacent guide coupling slots 254. Thus, in such embodiment, the guide coupling housing 250 is capable of retaining the guide proximal end 222P of up to seven energy guides (although only five energy guides 222A-222E are specifically shown in FIG. 2A). Alternatively, the guide coupling housing 250 can have a different number of guide coupling slots, i.e. greater than seven or fewer than seven, and/or the guide coupling slots 254 can be arranged in a different manner relative to one another.

The design of the multiplexer 228 can be varied depending on the requirements of the catheter system 200, the relative positioning of the energy guides 222A-222E, and/or to suit the desires of the user or operator of the catheter system 200. In the embodiment illustrated in FIG. 2A, the multiplexer 228 includes one or more of a multiplexer base 260, a multiplexer stage 262, a stage mover 264 (illustrated in phantom), a redirector 266, and coupling optics 268. Alternatively, the multiplexer 228 can include more components or fewer components than those specifically illustrated in FIG. 2A.

During use of the catheter system 200, the multiplexer base 260 is fixed in position relative to the energy source 224 and the energy guides 222A-222E. Additionally, in this embodiment, the multiplexer stage 262 is movably supported on the multiplexer base 260. More particularly, the stage mover 264 is configured to move the multiplexer stage 262 relative to the multiplexer base 260. As shown in FIG. 2A, the redirector 266 and the coupling optics 268 are mounted on and/or retained by the multiplexer stage 262. Thus, movement of the multiplexer stage 262 relative to the multiplexer base 260 results in corresponding movement of the redirector 266 and the coupling optics 268 relative to the fixed multiplexer base 260. Further, with the energy guides 222A-222E being fixed in position relative to the multiplexer base 260, movement of the multiplexer stage 262 results in corresponding movement of the redirector 266 and the coupling optics 268 relative to the energy guides 222A-222E.

In various embodiments, the multiplexer 228 is configured to precisely align the coupling optics 268 with each of the energy guides 222A-222E such that the source beam 224A generated by the energy source 224 can be precisely directed and focused by the multiplexer 228 as a corresponding guide beam 224B to each of the energy guides 222A-222E. In its simplest form, as shown in FIG. 2A, the multiplexer 228 uses a precision mechanism, i.e. the stage mover 264, to translate the coupling optics 268 along a linear path. This approach requires a single degree of freedom. In certain embodiments, the linear translation mechanism, i.e. the stage mover 264, and/or the multiplexer stage 262 can be equipped with mechanical stops so that the coupling optics 268 can be precisely aligned with the position of each of the energy guides 222A-222E in any desired firing sequence and/or pattern (whether predetermined or otherwise). Alternatively, the stage mover 264 can be electronically controlled to line the beam path of the guide beam 224B in any desired firing sequence and/or pattern with each individual energy guide 222A-222E that is retained, in part, within the guide coupling housing 250.

As noted above, the multiplexer stage 262 is configured to carry the necessary optics, e.g., the redirector 266 and the coupling optics 268, to direct and focus the light energy generated by the energy source 224 onto each energy guide 222A-222E for optimal coupling. With such design, the low divergence of the guide beam 224A over the short distance of motion of the translated multiplexer stage 262 has minimum impact on coupling efficiency to the energy guide 222A-222E.

During operation, the stage mover 264 drives the multiplexer stage 262 to align the beam path of the guide beam 224B with a selected energy guide 222A-222E and then the system controller 226 fires the energy source 224 in pulsed or semi-CW mode. The stage mover 264 then steps the multiplexer stage 262 to the next stop, i.e. to the next desired energy guide 222A-222E, and the system controller 226 again fires the energy source 224. This process is repeated as desired so that light energy in the form of the guide beams 224B is directed onto each of the energy guides 222A-222E in a desired firing sequence and/or pattern. It is appreciated that the stage mover 264 can move the multiplexer stage 262 so that it is aligned with any of the energy guides 222A-222E, then the system controller 226 fires the energy source 224. In this manner, the multiplexer 228 can achieve a firing sequence through the energy guides 222A-222E in any desired firing pattern.

In this embodiment, the stage mover 264 can have any suitable design for purposes of moving the multiplexer stage 262 in a linear manner relative to the multiplexer base 260. More particularly, the stage mover 264 can be any suitable type of linear translation mechanism.

As shown in FIG. 2A, the catheter system 200 can further include an optical element 246, e.g., a reflecting or redirecting element such as a mirror, that reflects the source beam 224A from the energy source 224 so that the source beam 224A is directed toward the multiplexer 228. In one embodiment, as shown, the optical element 246 can be positioned along the beam path to redirect the source beam 224A by approximately 90 degrees so that the source beam 224A is directed toward the multiplexer 228. Alternatively, the optical elements 246 can redirect the source beam 224A by more than 90 degrees or less than 90 degrees. Still alternatively, the catheter system 200 can be designed without the optical elements 246, and the energy source 224 can direct the source beam 224A directly toward the multiplexer 228.

Additionally, in this embodiment, the source beam 224A being directed toward the multiplexer 228 initially impinges on the redirector 266, which is configured to redirect the source beam 224A toward the coupling optics 268. In some embodiments, the redirector 266 redirects the source beam 224A by approximately 90 degrees toward the coupling optics 268. Alternatively, the redirector 266 can redirect the source beam 224A by more than 90 degrees or less than 90 degrees toward the coupling optics 268. Thus, the redirector 266 that is mounted on the multiplexer stage 262 is configured to direct the source beam 224A through the coupling optics 268 so that individual guide beams 224B are focused into the individual energy guides 222A-222E in the guide coupling housing 250.

The coupling optics 268 can have any suitable design for purposes of focusing the individual guide beams 224B onto each of the energy guides 222A-222E. In one embodiment, the coupling optics 268 includes two lenses that are specifically configured to focus the individual guide beams 224B as desired. Alternatively, the coupling optics 268 can have another suitable design.

In certain non-exclusive alternative embodiments, the steering of the source beam 224A so that it is properly directed and focused onto each of the energy guides 222A-222E can be accomplished using mirrors that are attached to optomechanical scanners, X-Y galvanometers or other multi-axis beam steering devices.

Still alternatively, although FIG. 2A illustrates that the energy guides 222A-222E are fixed in position relative to the multiplexer base 260, in some embodiments, it is appreciated that the energy guides 222A-222E can be configured to move relative to coupling optics 268 that are fixed in position. In such embodiments, the guide coupling housing 250 itself would move. In one non-exclusive example, the guide coupling housing 250 can be carried by a linear translation stage, and the system controller 226 can control the linear translation stage to move in a stepped manner so that the energy guides 222A-222E are each aligned, in a desired firing sequence or pattern, with the coupling optics and the guide beams 224B. While such an embodiment can be effective, it is further appreciated that additional protection and controls would be required to make it safe and reliable as the guide coupling housing 250 moves relative to the coupling optics 268 of the multiplexer 228 during use.

FIG. 2B is a simplified schematic perspective view of a portion of the catheter system 200 and the multiplexer 228 illustrated in FIG. 2A. In particular, FIG. 2B illustrates another view of the guide coupling housing 250, with the guide coupling slots 254, that is configured to retain a portion of each of the energy guides 222A-222E; the optical element 246 that initially redirects the source beam 224A from the energy source 224 (illustrated in FIG. 2A) toward the multiplexer 228; and the multiplexer 228, including the multiplexer base 260, the multiplexer stage 262, the redirector 266 and the coupling optics 268, that receives the source beam 224A and then directs and focuses individual guide beams 224B in any desired firing sequence and/or pattern toward each of the energy guides 222A-222E. It is appreciated that the stage mover 264 is not illustrated in FIG. 2B for purposes of simplicity and ease of illustration.

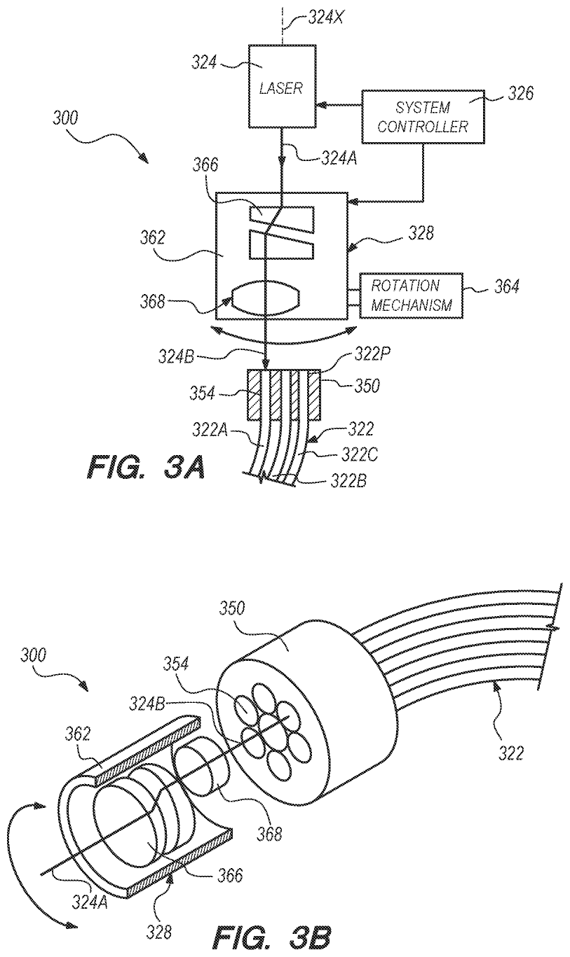

FIG. 3A is a simplified schematic top view of a portion of an embodiment of the catheter system 300 including another embodiment of the multiplexer 328. More particularly, FIG. 3A illustrates a plurality of energy guides, e.g., a first energy guide 322A, a second energy guide 322B and a third energy guide 322C, an energy source 324, a system controller 326, and the multiplexer 328 that receives light energy in the form of a source beam 324A from the energy source 324 and selectively and/or alternatively directs the light energy in the form of individual guide beams 324B in any desired firing sequence and/or pattern to each of the energy guides 322A-322C, i.e. under control of the system controller 326. The energy guides 322A-322C, the energy source 324 and the system controller 326 are substantially similar in design and function as described in detail herein above. Accordingly, such components will not be described in detail in relation to the embodiment illustrated in FIG. 3A. It is further appreciated that certain components of the system console 123 illustrated and described above in relation to FIG. 1, e.g., the power source 125 and the GUI 127, are not illustrated in FIG. 3A for purposes of simplicity and ease of illustration, but would typically be included in many embodiments.

As with previous embodiments, the multiplexer 328 is configured to receive light energy in the form of the source beam 324A, e.g., a single pulsed source beam, from the energy source 324 and selectively and/or alternatively direct the light energy in the form of individual guide beams 324B in any desired firing sequence and/or pattern to each of the energy guides 322A-322C. As such, as shown in FIG. 3A, the multiplexer 328 is operatively and/or optically coupled in optical communication to the energy guide bundle 322, i.e. to the plurality of energy guides 322A-322C.

Additionally, as illustrated, a guide proximal end 322P of each of the plurality of energy guides 322A-322C is retained within a guide coupling housing 350, i.e. within guide coupling slots 354 that are formed into the guide coupling housing 350. In various embodiments, the guide coupling housing 350 is configured to be selectively coupled to the system console 123 (illustrated in FIG. 1) so that the guide coupling slots 354, and thus the energy guides 322A-322C, are maintained in a desired fixed position relative to the multiplexer 328 during use of the catheter system 300.

Referring now to FIG. 3B, FIG. 3B is a simplified schematic perspective view of a portion of the catheter system 300 and the multiplexer 328 illustrated in FIG. 3A. As shown in FIG. 3B, the guide coupling housing 350 can be substantially cylindrical-shaped. It is appreciated that the guide coupling housing 350 can have any suitable number of guide coupling slots 354, which can be positioned and/or oriented relative to one another in any suitable manner, e.g., to best align the guide coupling slots 354 and thus the energy guides 322A-322C of the energy guide bundle 322 relative to the multiplexer 328. In the embodiment illustrated in FIG. 3B, the guide coupling housing 350 includes seven guide coupling slots 354 that are arranged in a circular and/or hexagonal packed pattern. Thus, in such embodiment, the guide coupling housing 350 is capable of retaining the guide proximal end of up to seven energy guides. Alternatively, the guide coupling housing 350 can have a different number of guide coupling slots, i.e. greater than seven or less than seven, and/or the guide coupling slots 354 can be arranged in a different manner relative to one another, e.g., in another suitable circular periodic pattern.

Returning to FIG. 3A, in this embodiment, the multiplexer 328 includes one or more of a multiplexer stage 362, a stage mover 364, a redirector 366, and coupling optics 368. Alternatively, the multiplexer 328 can include more components or fewer components than those specifically illustrated in FIG. 3A.

As shown in the embodiment illustrated in FIG. 3A, the stage mover 364 is configured to move the multiplexer stage 362 in a rotational manner. More particularly, in this embodiment, the multiplexer stage 362 and/or the stage mover 364 requires a single rotational degree of freedom. Additionally, as shown, the multiplexer stage 362 and the guide coupling housing 350 are aligned on a central axis 324X of the energy source 324. As such, the multiplexer stage 362 is configured to be rotated by the stage mover 364 about the central axis 324X.

The redirector 366 and the coupling optics 368 are mounted on and/or retained by the multiplexer stage 362. During use of the catheter system 300, the source beam 324A is initially directed toward the multiplexer 328, i.e. the multiplexer stage 362, along the central axis 324X of the energy source 324. Subsequently, the redirector 366 is configured to deviate the source beam 324A a fixed distance laterally, i.e. off the central axis 324X of the energy source 324, such that the source beam 324A is directed in a direction that is substantially parallel to and spaced apart from the central axis 324X. More specifically, the redirector 366 deviates the source beam 324A to coincide with the radius of the circular pattern of the energy guides 322A-322C in the guide coupling housing 350. As the multiplexer stage 362 is rotated, the source beam 324A that is directed through the redirector 366 traces out a circular path.

It is appreciated that the redirector 366 can have any suitable design. For example, in certain non-exclusive alternative embodiments, the redirector 366 can be provided in the form of an anamorphic prism pair, a pair of wedge prisms, or a pair of close-spaced right angle mirrors or prisms. Alternatively, the redirector 366 can include another suitable configuration of optics in order to achieve the desired lateral beam offset.

Additionally, as noted, the coupling optics 368 are also mounted on and/or retained by the multiplexer stage 362. As with the previous embodiments, the coupling optics 368 are configured to focus the individual guide beams 324B onto each of the energy guides 322A-322C in the energy guide bundle 322 retained, in part, within the guide coupling housing 350 for optimal coupling.

As noted above, the multiplexer 328 is configured to precisely align the coupling optics 368 with each of the energy guides 322A-322C such that the source beam 324A generated by the energy source 324 can be precisely directed and focused by the multiplexer 328 as a corresponding guide beam 324B to each of the energy guides 322A-322C. In certain embodiments, the stage mover 364 and/or the multiplexer stage 362 can be equipped with mechanical stops so that the coupling optics 368 can be precisely aligned with the position of each of the energy guides 322A-322C in any desired firing sequence and/or pattern. Alternatively, the stage mover 364 can be electronically controlled, e.g., using stepper motors or a piezo-actuated rotational stage, to line the beam path of the guide beam 324B in any desired firing sequence and/or pattern with each individual energy guide 322A-322C that is retained, in part, within the guide coupling housing 350.

During use of the catheter system 300, the stage mover 364 drives the multiplexer stage 362 to couple the guide beam 324B with a selected energy guide 322A-322C and then the system controller 326 fires the energy source 324 in pulsed or semi-CW mode. The stage mover 364 then steps the multiplexer stage 362 angularly to the next stop, i.e. to the next desired energy guide 322A-322C, and the system controller 326 again fires the energy source 324. This process is repeated as desired so that light energy in the form of the guide beams 324B is directed onto each of the energy guides 322A-322C in a desired firing sequence and/or pattern. It is appreciated that the stage mover 364 can move the multiplexer stage 362 so that it is aligned with any of the energy guides 322A-322C, then the system controller 326 fires the energy source 324. In this manner, the multiplexer 328 can achieve a particular firing sequence through the energy guides 322A-322C or fire in any desired firing sequence or pattern relative to the energy guides 322A-322C.

In this embodiment, the stage mover 364 can have any suitable design for purposes of moving the multiplexer stage 362 in a rotational manner about the central axis 324X. More particularly, the stage mover 364 can be any suitable type of rotational mechanism.

Alternatively, although FIG. 3A illustrates that the energy guides 322A-322C are fixed in position relative to the multiplexer stage 362, in some embodiments, it is appreciated that the energy guides 322A-322C can be configured to move, e.g., rotate, relative to coupling optics 368 that are fixed in position. In such embodiments, the guide coupling housing 350 itself would move, e.g., the guide coupling housing 350 can be rotated about the central axis 324X, and the system controller 326 can control the rotational stage to move in a stepped manner so that the energy guides 322A-322C are each aligned, in a desired firing sequence and/or pattern, with the coupling optics and the guide beams 324B. In such embodiment, the guide coupling housing 350 would not be continuously rotated, but would be rotated a fixed number of degrees and then counter-rotated to avoid the winding of the energy guides 322A-322C.

Returning again to FIG. 3B, another view of the guide coupling housing 350 is shown. FIG. 3B illustrates the guide coupling slots 354, that are configured to retain a portion of each of the energy guides, and the multiplexer 328, including the multiplexer stage 362, the redirector 366 and the coupling optics 368, that receives the source beam 324A and then directs and focuses individual guide beams 324B in any desired firing sequence and/or firing pattern toward each of the energy guides. It is appreciated that the stage mover 364 is not illustrated in FIG. 3B for purposes of simplicity and ease of illustration.

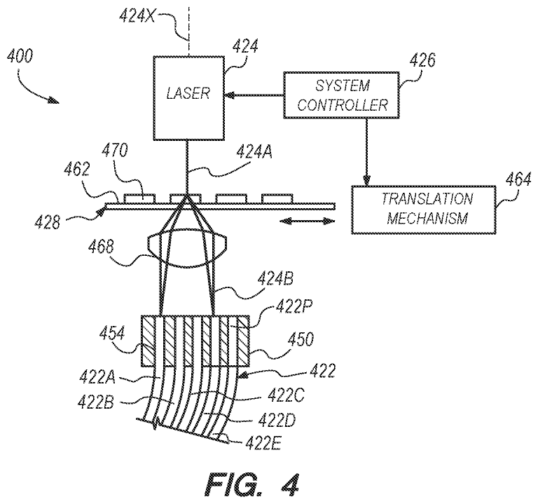

FIG. 4 is a simplified schematic top view of a portion of the catheter system 400 and still another embodiment of the multiplexer 428. More particularly, FIG. 4 illustrates a plurality of energy guides, e.g., a first energy guide 422A, a second energy guide 422B, a third energy guide 422C, a fourth energy guide 422D and a fifth energy guide 422E, an energy source 424, a system controller 426, and the multiplexer 428 that receives light energy in the form of a source beam 424A from the energy source 424 and selectively and/or alternatively directs the light energy in the form of individual guide beams 424B in any desired firing sequence and/or pattern to each of the energy guides 422A-422E, i.e. under control of the system controller 426. The energy guides 422A-422E, the energy source 424 and the system controller 426 are substantially similar in design and function as described in detail herein above. Accordingly, such components will not be described in detail in relation to the embodiment illustrated in FIG. 4. It is further appreciated that certain components of the system console 123 illustrated and described above in relation to FIG. 1, e.g., the power source 125 and the GUI 127, are not illustrated in FIG. 4 for purposes of simplicity and ease of illustration, but would typically be included in many embodiments.

As noted above, the multiplexer 428 is configured to receive light energy in the form of the source beam 424A, e.g., a single pulsed source beam, from the energy source 424 and selectively and/or alternatively direct the light energy in the form of individual guide beams 424B in any desired firing sequence and/or pattern to each of the energy guides 422A-422E. As such, as shown in FIG. 4, the multiplexer 428 is operatively and/or optically coupled in optical communication to the energy guide bundle 422, i.e. to the plurality of energy guides 422A-422E.

Additionally, as illustrated, a guide proximal end 422P of each of the plurality of energy guides 422A-422E is retained within a guide coupling housing 450, i.e. within guide coupling slots 454 that are formed into the guide coupling housing 450. In various embodiments, the guide coupling housing 450 is configured to be selectively coupled to the system console 123 (illustrated in FIG. 1) so that the guide coupling slots 454, and thus the energy guides 422A-422E, are maintained in a desired fixed position relative to the multiplexer 428 during use of the catheter system 400. It is appreciated that the guide coupling housing 450 can have any suitable number of guide coupling slots 454. In the embodiment illustrated in FIG. 4, five guide coupling slots 454 are visible within the guide coupling housing 450. Thus, in such embodiment, the guide coupling housing 450 is capable of retaining the guide proximal end 422P of up to five energy guides. Alternatively, the guide coupling housing 450 can have a different number of guide coupling slots 454, i.e. greater than five or less than five guide coupling slots 454.

In the embodiment illustrated in FIG. 4, the multiplexer 428 includes one or more of a multiplexer stage 462, a stage mover 464, one or more diffractive optical elements 470 (or “DOE”), and coupling optics 468. Alternatively, the multiplexer 428 can include more components or fewer components than those specifically illustrated in FIG. 4.

As shown, the diffractive optical elements 470 are mounted on and/or retained by the multiplexer stage 462. Additionally, the stage mover 464 is configured to move the multiplexer stage 462, e.g., translationally, such that each of the one or more diffractive optical elements 470 are selectively and/or alternatively positioned in the beam path of the source beam 424A from the energy source 424.

During use of the catheter system 400, each of the one or more diffractive optical elements 470 is configured to separate the source beam 424A into one, two, three or more individual guide beams 424B. It is appreciated that the diffractive optical elements 470 can have any suitable design. For example, in certain non-exclusive embodiments, the diffractive optical elements 470 can be created using arrays of micro-prisms, micro-lenses, or other patterned diffractive elements.

It is appreciated that there are many possible physical configurations, patterns or setups to organize the energy guides 422A-422E in the guide coupling housing 450 using this approach. One such configuration for the energy guides 422A-422E within the guide coupling housing 450 would be a hexagonal, closely-packed configuration, somewhat similar to that illustrated in FIGS. 3A and 3B. Alternatively, the energy guides 422A-422E within the guide coupling housing 450 could also be arranged in a square, rectangular, triangular, pentagonal, linear, circular, or any other suitable geometric or non-geometric configuration.

As shown in FIG. 4, the guide coupling housing 450 can be aligned on the central axis 424X of the energy source 424, with the diffractive optical elements 470 mounted on the multiplexer stage 462 being inserted along the beam path between the energy source 424 and the guide coupling housing 450. Additionally, as illustrated, the coupling optics 468 are also positioned along the central axis 424X of the energy source 424, and the coupling optics are positioned between the diffractive optical elements 470 and the guide coupling housing 450.

During operation, the source beam 424A impinging on one of the plurality of diffractive optical elements 470 splits the source beam 424A into two or more deviated beams, i.e. two or more guide beams 424B. These guide beams 424B are, in turn, directed and focused by the coupling optics 468 down onto the individual energy guides 422A-422E that are retained in the guide coupling housing 450. In one configuration, the diffractive optical element 470 would split the source beam 424A into as many energy guides as are present within the single-use device. In such configuration, the power in each guide beam 424B is based on the number of guide beams 424B that are generated from the single source beam 424A minus scattering and absorption losses. Alternatively, the diffractive optical element 470 can be configured to split the source beam 424A so that guide beams 424B are directed into any single energy guide or any selected multiple energy guides. Thus, the multiplexer stage 462 can be configured to retain a plurality of diffractive optical elements 470, e.g., with multiple diffractive optical element patterns etched on a single plate, to provide options for the user or operator for coupling the guide beams 424B to the desired number and pattern of energy guides. In such embodiments, the desired firing sequence can be achieved by moving the multiplexer stage 462 with the stage mover 464, e.g., translationally, so that the desired diffractive optical element 470 is positioned in the beam path of the source beam 424A between the energy source 424 and the coupling optics 468.

As with the previous embodiments, the coupling optics 468 can have any suitable design for purposes of focusing the individual guide beams 424B, or multiple guide beams 424B simultaneously, onto the desired energy guides 422A-422E

FIG. 5 is a simplified schematic top view of a portion of the catheter system 500 and yet another embodiment of the multiplexer 528. More particularly, FIG. 5 illustrates a plurality of energy guides, e.g., a first energy guide 522A, a second energy guide 522B and a third energy guide 522C, an energy source 524, a system controller 526, and the multiplexer 528 that receives light energy in the form of a source beam 524A from the energy source 524 and selectively and/or alternatively directs the light energy in the form of individual guide beams 524B in any desired firing sequence and/or pattern to each of the energy guides 522A-522C, i.e. under control of the system controller 526. The energy guides 522A-522C, the energy source 524 and the system controller 526 are substantially similar in design and function as described in detail herein above. Accordingly, such components will not be described in detail in relation to the embodiment illustrated in FIG. 5. It is further appreciated that certain components of the system console 123 illustrated and described above in relation to FIG. 1, e.g., the power source 125 and the GUI 127, are not illustrated in FIG. 5 for purposes of simplicity and ease of illustration, but would typically be included in many embodiments.

As noted above, the multiplexer 528 is configured to receive light energy in the form of the source beam 524A, e.g., a single pulsed source beam, from the energy source 524 and selectively and/or alternatively direct the light energy in the form of individual guide beams 524B in any desired firing sequence and/or pattern to each of the energy guides 522A-522C. As such, as shown in FIG. 5, the multiplexer 528 is operatively and/or optically coupled in optical communication to the plurality of energy guides 522A-522C.

However, as illustrated in FIG. 5, the multiplexer 528 has a different design than any of the previous embodiments. In some embodiments, it may be desirable to design the multiplexer 528 to receive the source beam 524A from a single energy source 524 and selectively and/or alternatively direct the light energy in the form of individual guide beams 524B in any desired firing sequence and/or pattern to each of the energy guides 522A-522C in a manner that is easily reconfigurable and that does not involve moving parts. For example, using an acousto-optic deflector (AOD) as the multiplexer 528 can allow the entire output of a single energy source 524, e.g., a single laser, to be directed into a plurality of individual energy guides 522A-522C. The guide beam 524B can be retargeted to a different energy guide 522A-522C within microseconds by simply changing the driving frequency input into the multiplexer 528 (the AOD), and with a pulsed laser such as a Nd:YAG, this switching can easily occur between pulses. In such embodiments, the deflection angle (Θ) of the multiplexer 528 can be defined as follows:
Deflection angle (Θ)=Λf/v where
Λ=Optical Wavelength
f=acoustic drive frequency
v=speed of sound in modulator

As shown in FIG. 5, the source beam 524A is directed from the energy source 524 toward the multiplexer 528, and is subsequently redirected due to the generated deflection angle as a desired guide beam 524B to each of the energy guides 522A-522C. More specifically, as illustrated, when the multiplexer 528 generates a first deflection angle for the source beam 524A, a first guide beam 524B1 is directed to the first energy guide 522A; when the multiplexer 528 generates a second deflection angle for the source beam 524A, a second guide beam 524B2 is directed to the second energy guide 522B; and when the multiplexer 528 generates a third deflection angle for the source beam 524A, a third guide beam 524B3 is directed to the third energy guide 522C. It is appreciated that, as illustrated, any desired deflection angle can include effectively no deflection angle at all, i.e. the guide beam 524B can be directed to continue along the same axial beam path as the source beam 524A.

In this embodiment, the multiplexer 528 (AOD) includes a transducer 572 and an absorber 574 that cooperate to generate the desired driving frequency that can, in turn, generate the desired deflection angle so that the source beam 524A is redirected as the desired guide beam 524B toward the desired energy guide 522A-522C. More particularly, the multiplexer 528 is configured to spatially control the source beam 524A. In the operation of the multiplexer 528, the power driving the acoustic transducer 572 is kept on, at a constant level, while the acoustic frequency is varied to deflect the source beam 524A to different angular positions that define the guide beams 524B1-524B3. Thus, the multiplexer 528 makes use of the acoustic frequency-dependent diffraction angle, such as described above.

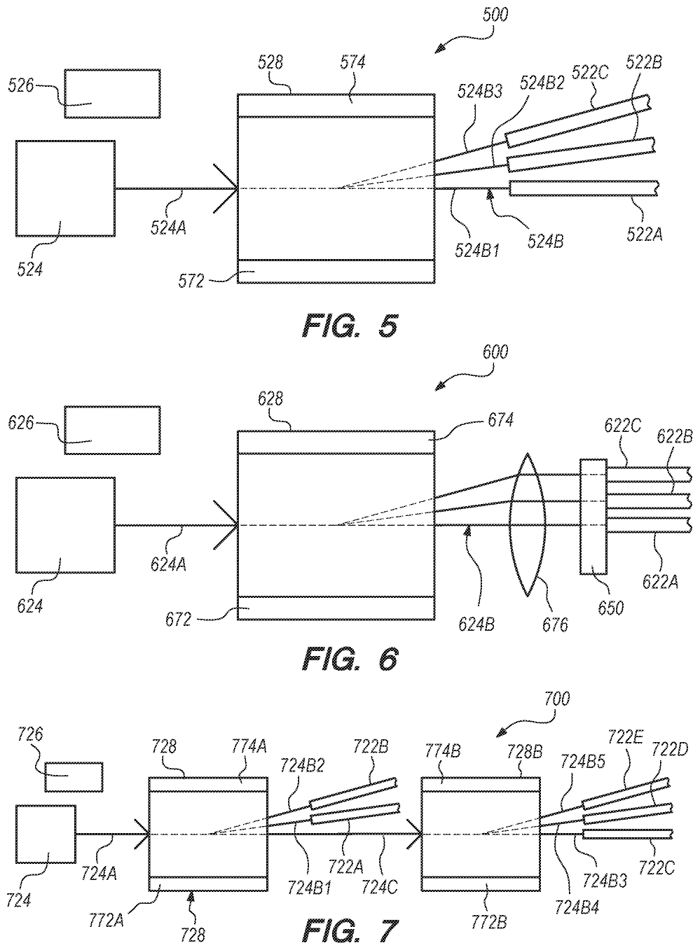

FIG. 6 is a simplified schematic top view of a portion of the catheter system 600 and still another embodiment of the multiplexer 628. More particularly, FIG. 6 illustrates a plurality of energy guides, e.g., a first energy guide 622A, a second energy guide 622B and a third energy guide 622C, an energy source 624, a system controller 626, and the multiplexer 628 that receives light energy in the form of a source beam 624A, e.g., a single pulsed source beam, from the energy source 624 and selectively and/or alternatively directs the light energy in the form of individual guide beams 624B in any desired firing sequence and/or pattern to each of the energy guides 622A-622C, i.e. under control of the system controller 626. The energy guides 622A-622C, the energy source 624 and the system controller 626 are substantially similar in design and function as described in detail herein above. Accordingly, such components will not be described in detail in relation to the embodiment illustrated in FIG. 6. It is further appreciated that certain components of the system console 123 illustrated and described above in relation to FIG. 1, e.g., the power source 125 and the GUI 127, are not illustrated in FIG. 6 for purposes of simplicity and ease of illustration, but would typically be included in many embodiments.

It is appreciated that the multiplexer 628 illustrated in FIG. 6 is substantially similar to the multiplexer 528 illustrated and described in relation to FIG. 5. For example, as shown in FIG. 6, the multiplexer 628 again includes a transducer 672 and an absorber 674 that cooperate to generate the desired driving frequency that can, in turn, generate the desired deflection angle so that the source beam 624A is redirected as the desired guide beam 624B toward the desired energy guide 622A-622C. However, in this embodiment, the multiplexer 628 further includes an optical element 676 that is usable to transform the angular separation between the guide beams 624B into a linear offset.

In some embodiments, in order to improve the angular resolution and the efficiency of the catheter system 600, the input laser 624 should be collimated with a diameter close to filling the aperture of the multiplexer 628 (the AOD). The smaller the divergence of the input, the greater number of discrete outputs can be generated. The angular resolution of such a device is quite good, but the total angular deflection is limited. To allow a sufficient number of energy guides 622A-622C of finite size to be accessed by a single energy source 624 and a single source beam 624A, there are a number of means to improve the separation of the different output. For example, as shown in FIG. 6, after the individual guide beams 624B separate, the optical element 676, e.g., a lens, can be used to transform the angular separation between the guide beams 624B into a linear offset, and can be used to direct the guide beams 624B into closely spaced energy guides 622A-622C, e.g., when the energy guides 622A-622C are held in close proximity to one another within a guide coupling housing 650. Additionally, folding mirrors can be used to allow adequate propagation distance to separate the different beam paths of the guide beams 624B within a limited volume.

FIG. 7 is a simplified schematic top view of a portion of the catheter system 700 and still yet another embodiment of the multiplexer 728. More particularly, FIG. 7 illustrates a plurality of energy guides, e.g., a first energy guide 722A, a second energy guide 722B, a third energy guide 722C, a fourth energy guide 722D and a fifth energy guide 722E, an energy source 724, a system controller 726, and the multiplexer 728 that receives light energy in the form of a source beam 724A, e.g., a single pulsed source beam, from the energy source 724 and selectively and/or alternatively directs the light energy in the form of individual guide beams 724B in any desired firing sequence and/or pattern to each of the energy guides 722A-722E, i.e. under control of the system controller 726. The energy guides 722A-722E, the energy source 724 and the system controller 726 are substantially similar in design and function as described in detail herein above. Accordingly, such components will not be described in detail in relation to the embodiment illustrated in FIG. 7. It is further appreciated that certain components of the system console 123 illustrated and described above in relation to FIG. 1, e.g., the power source 125 and the GUI 127, are not illustrated in FIG. 7 for purposes of simplicity and ease of illustration, but would typically be included in many embodiments.

It is appreciated that the manner for multiplexing the source beam 724A into multiple guide beams 724B illustrated in FIG. 7 is somewhat similar to how the source beam 524 was multiplexed into multiple guide beams 524B as illustrated and described in relation to FIG. 5. However, in this embodiment, the multiplexer 728 includes a pair of acousto-optic deflectors (AODs), i.e. a first acousto-optic deflector 728A and a second acousto-optic deflector 728B, that are positioned in series with one another. With such design, the multiplexer 728 may be able to access additional energy guides. Additionally, it is further appreciated that the multiplexer 728 can include more than two acousto-optic deflectors, if desired, to be able to access even more energy guides.

In the embodiment shown in FIG. 7, the source beam 724A is initially directed toward the first AOD 728A. The first AOD 728A is utilized to deflect the source beam 724A to generate a first guide beam 724B1 that is directed toward the first energy guide 722A, and a second guide beam 724B2 that is directed toward the second energy guide 722B2. Additionally, the first AOD 728A also allows an undeviated beam to be transmitted through the first AOD 728A as a transmitted beam 724C that is directed toward the second AOD 728B. Subsequently, the second AOD 728B is utilized to deflect the transmitted beam 724C, as desired, to generate a third guide beam 724B3 that is directed toward the third energy guide 722C, a fourth guide beam 724B4 that is directed toward the fourth energy guide 722D, and a fifth guide beam 724B5 that is directed toward the fifth energy guide 722B5.

Additionally, each AOD 728A, 728B can be designed in a similar manner to those described in greater detail above. For example, the first AOD 728A can include a first transducer 772A and a first absorber 774A that cooperate to generate the desired driving frequency that can, in turn, generate the desired deflection angle so that the source beam 724A is redirected as desired; and the second AOD 728B can include a second transducer 772B and a second absorber 774B that cooperate to generate the desired driving frequency that can, in turn, generate the desired deflection angle so that the transmitted beam 724C is redirected as desired. Alternatively, the first AOD 728A and/or the second AOD 728B can have another suitable design.

FIG. 8A is a simplified schematic side view of a portion of another embodiment of the catheter system 800. More specifically, as shown in FIG. 8A, the catheter system 800 includes at least a balloon 804, a catheter shaft 810, a guidewire lumen 818, and a plurality of energy guides 822 which are spaced apart from one another about the circumference of the guidewire lumen 818. The balloon 804, the catheter shaft 810, the guidewire lumen 818 and the plurality of energy guides 822 are generally similar in design and operation to what has been described in detail herein above. Thus, the balloon 804, the catheter shaft 810, the guidewire lumen 818 and the plurality of energy guides 822 will not be described in detail again in relation to the embodiment shown in FIG. 8A.

As with embodiments described in detail above, the catheter system 800, e.g., including the energy source 124 (illustrated in FIG. 1) and/or the multiplexer 128 (illustrated in FIG. 1), can be configured and controlled, i.e. by the system controller 126 (illustrated in FIG. 1), to selectively and/or separately power each of the plurality of energy guides 822 in any desired firing sequence, pattern, order, firing rate and/or firing duration in order to impart pressure onto and induce fractures in vascular lesions. Additionally, as noted above, it is appreciated that although each of the plurality of energy guides 822 can be powered separately in any desired firing sequence, pattern, order, firing rate and/or firing duration, sets and/or subsets of the plurality of energy guides 822 can also be powered at any given point in time. Each set or subset of the plurality of energy guides 822 can include one or more of the plurality of energy guides 822. Thus, at any given point in time, power can be directed to one or more of the plurality of energy guides 822 to alternatively create a first firing sequence, a second firing sequence, a third firing sequence, a fourth firing sequence, etc. Moreover, although not required, one or more of the firing sequences of the energy guides 822 in such sets and subsets of the plurality of energy guides 822 can be different than any or all of the other firing sequences of the energy guides 822.

FIG. 8B is a simplified schematic cross-sectional view of the portion of the catheter system 800 taken on line B-B in FIG. 8A. More particularly, FIG. 8B again illustrates the balloon 804, the catheter shaft 810, the guidewire lumen 818, and the plurality of energy guides 822 that can be included within this embodiment of the catheter system 800. Further, as shown, FIG. 8B illustrates that this particular non-exclusive embodiment of the catheter system 800 includes eight energy guides, including a first energy guide 822A, a second energy guide 822B, a third energy guide 822C, a fourth energy guide 822D, a fifth energy guide 822E, a sixth energy guide 822F, a seventh energy guide 822G, and an eighth energy guide 822H. It is understood, however, that any suitable number of energy guides can be used. Additionally, in FIG. 8B, the energy guides 822A-822H are uniformly separated by about 45 degrees from one another around the circumference of the guidewire lumen 818. However, it is appreciated that the energy guides 822A-822H need not be uniformly separated from one another, i.e. the energy guides 822A-822H can be non-uniformly separated from one another, around the circumference of the guidewire lumen 818.

Further, as provided herein, each of the energy guides 822A-822H includes a guide distal end 880 (illustrated in FIG. 8A) that can be positioned in any suitable or desired longitudinal position relative to a length 842 of the balloon 804 and/or a length of the guidewire lumen 818 to more effectively and precisely impart pressure waves for purposes of disrupting the vascular lesions at the treatment site 106 (illustrated in FIG. 1). For example, also referring to FIG. 8A, the first energy guide 822A can include a first guide distal end 880 that is positioned at a first longitudinal position relative to the length 842 of the balloon 804; the second energy guide 822B can include a second guide distal end 880 that is positioned at a second longitudinal position relative to the length 842 of the balloon 804; the third energy guide 822C can include a third guide distal end 880 that is positioned at a third longitudinal position relative to the length 842 of the balloon 804; the fourth energy guide 822D can include a fourth guide distal end 880 that is positioned at a fourth longitudinal position relative to the length 842 of the balloon 804; the fifth energy guide 822E can include a fifth guide distal end 880 that is positioned at a fifth longitudinal position relative to the length 842 of the balloon 804; the sixth energy guide 822F can include a sixth guide distal end 880 that is positioned at a sixth longitudinal position relative to the length 842 of the balloon 804; the seventh energy guide 822G can include a seventh guide distal end 880 that is positioned at a seventh longitudinal position relative to the length 842 of the balloon 804; and the eighth energy guide 822H can include an eighth guide distal end 880 that is positioned at an eighth longitudinal position relative to the length 842 of the balloon 804.

It is appreciated that, in alternative embodiments, each of the longitudinal positions of the guide distal ends 880 relative to the length 842 of the balloon 804 can be different than one another, or two or more of the longitudinal positions of the guide distal ends 880 relative to the length 842 of the balloon 804 can be the same as one another. Additionally, as noted above, although each of the energy guides 822A-822H is shown as being positioned substantially directly adjacent to the guidewire lumen 818, it is recognized that a portion of the energy guide 822A-822H, e.g., the guide distal end 880, can be spaced apart from the guidewire lumen 818. For example, the guide distal end 880 of any of the energy guides 822A-822H can be located at any suitable position laterally between the guidewire lumen 818 and the balloon wall 830 of the balloon 804.

As noted above, it is possible to fire individual energy guides 822A-822H, and/or sets or subsets of the energy guides 822A-822H, to achieve a desired firing sequence or pattern that could be more effective at disrupting localized calcified lesions. More specifically, the system controller 126 can control the energy source 124 and/or the multiplexer 128 so that individual guide beams 124B (illustrated in FIG. 1) can be directed to each of the energy guides 822A-822H, or sets or subsets of the energy guides 822A-822H, in any desired firing sequence, pattern, order, firing rate and/or firing duration to achieve a greater degree of disruption of the calcified lesions. For example, with eight energy guides 822A-822H that are arranged in a linear pattern with angular orientation spiraling around the guidewire lumen 818, the system controller 126 can control the firing sequence of the light energy from the energy source 124 to each of the energy guides 822A-822H in any desired predetermined or non-predetermined manner.

For example, in one non-exclusive embodiment, the system controller 126 can direct individual guide beams 124B to each of the first energy guide 822A and the eighth energy guide 822H in a first set of energy guides, then having individual guide beams 124B directed to each of the second energy guide 822B and the seventh energy guide 822G in a second set of energy guides, followed by individual guide beams 124B directed to each of the third energy guide 822C and the sixth energy guide 822F in a third set of energy guides, and finally having individual guide beams 124B directed to each of the fourth energy guide 822D and the fifth energy guide 822E in a fourth set of energy guides. This example of a firing sequence and/or firing pattern generates an advancing wavefront that would converge from both ends of the balloon 804 toward a specific region such as the treatment site 106, located between the guide distal ends 880 of the fourth energy guide 822D and the fifth energy guide 822E, and can thereby more effectively disrupt a lesion at that location.

As used herein, the term “advancing wavefront” is intended to mean a series of guide beams that are directed to one or more energy guides so that an overall pattern is generated wherein light energy causing pressure waves is generally moving toward the treatment site 106. Conversely, a “retreating wavefront” would in effect be somewhat the opposite of an advancing wavefront. In other words, a series of guide beams are directed to one or more energy guides so that an overall pattern is generated wherein light energy causing pressure waves is generally moving away from the treatment site 106. It is understood that those skilled in the art would understand the retreating wavefront to operate in substantially the opposite manner as the advancing wavefront.

In another non-exclusive embodiment, the system controller 126 can direct individual guide beams 124B to each of the third energy guide 822C and the seventh energy guide 822G in a first set of energy guides, followed by individual guide beams 124B directed to each of the fourth energy guide 822D and the sixth energy guide 822F in a second set of energy guides, and finally having an individual guide beam 124B directed to the fifth energy guide 822E in a third set of energy guides. This example of a firing sequence and/or firing pattern generates an advancing wavefront that would converge from both ends of the balloon 804 toward a specific region (such as the treatment site 106, in one embodiment) located at a longitudinal position, i.e. the fifth longitudinal position, within the balloon 804 near the guide distal end 880 of the fifth energy guide 822E.

In still another non-exclusive embodiment, the system controller 126 can control the energy source 124 and/or the multiplexer 128 to generate a somewhat different advancing wavefront along substantially a full length 842 of the balloon 804. In this embodiment, the system controller 126 can control the energy source 124 and/or the multiplexer 128 to sequentially direct individual guide beams 124B to the first energy guide 822A, the second energy guide 822B, and the third energy guide 822C in a first sequence. Next, the system controller 126 can control the energy source 124 and/or the multiplexer 128 to sequentially direct individual guide beams 124B to the second energy guide 822B, the third energy guide 822C, and the fourth energy guide 822D in a second sequence. This type of sequencing can continue in a similar manner, (e.g., third, fourth, then fifth in a third sequence; fourth, fifth, then sixth in a fourth sequence; fifth, sixth, then seventh in a fifth sequence, etc.), until a last step when the system controller 126 controls the energy source 124 and/or the multiplexer 128 to sequentially direct individual guide beams 124B to the sixth energy guide 822F, the seventh energy guide 822G, and the eighth energy guide 822H in a sixth sequence.

In various embodiments, the advancing wavefront need not necessarily solely advance toward the treatment site 106, but may represent a wavefront that advances toward the treatment site 106 and subsequently continues past the treatment site 106 (in any suitable direction).

Further, or in the alternative, the system controller 126 can also control the energy source 124 and/or the multiplexer 128 to create an advancing series of energy waves along the length 842 of the balloon 804 for creating a shear wave in the lesion at the treatment site 106. In such applications, as individual guide beams 124B are directed to specific energy guides 822 in a specific sequence, the pressure waves will advance in the direction of activation. As new bubbles are created ahead of collapsing ones, it would be possible to create a shearing force at the balloon wall 830. The localized force on the leading edge of the spherical wavefront impinging at an angle relative to the balloon wall 830 that is non-normal creates a highly concentrated, localized shearing force. This could have a greater effect in cracking calcified lesions compared to simply hitting the walls through the length 842 of the balloon 804 with one radially directed pressure wave, which can expand the whole cross-section of the balloon 804 creating hoop stress.

Still further, in certain embodiments, it may be desirable to have the guide distal end 880 of more than one energy guide 822A-822H be positioned at the same longitudinal position relative to the length 842 of the balloon 804, and the system controller 126 can control the energy source 124 and/or the multiplexer 128 to fire each of such energy guides 822A-822H substantially simultaneously to generate pressure waves fully about the balloon 804 at such longitudinal position.

It is also appreciated that the foregoing examples of embodiments describing and/or illustrating particular firing sequences or patterns are provided as representative examples only, and are not intended to be limiting in any manner. In fact, it is further appreciated that an unlimited number of different such firing sequences and/or patterns can be achieved utilizing the disclosure provided herein. It is appreciated that with any of the embodiments shown, described and/or achievable using the disclosure herein, that the firing rate can be controlled so that the firing rate increases or decreases over time. Further, the firing rate can be controlled so that the firing rate increases or decreases depending upon the specific energy guides to which light energy is being directed.

It is recognized that with the designs provided herein, any desired firing sequence and/or pattern can be achieved. The types of firing sequences and/or patterns that can be achieved can be based at least in part on the number of energy guides, the axial and longitudinal positioning of each of the energy guides within the balloon, the energy level of the firing of each of the energy guides, the rate of firing, etc. It is understood that by controlling these and any other suitable parameters, an advancing wavefront resulting in a gradual or abrupt disruption of the calcification of a vascular lesion can occur. With these designs, the likelihood of success for adequate and/or satisfactory disruption of the calcification in a vascular lesion and/or heart valve is increased.

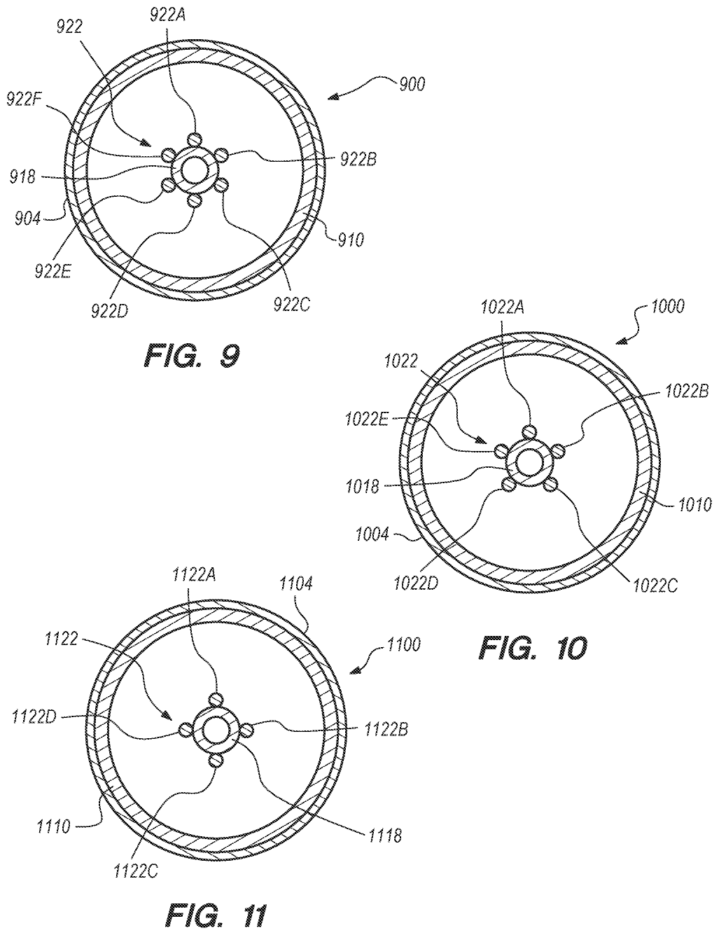

FIG. 9 is a simplified schematic cross-sectional view of another embodiment of the catheter system 900. The catheter system 900 illustrated in FIG. 9 is similar to the catheter system 800 illustrated in FIGS. 8A and 8B, except that the catheter system 900 in FIG. 9 includes a different number of energy guides. More particularly, FIG. 9 illustrates that the catheter system 900 includes at least a balloon 904, a catheter shaft 910, a guidewire lumen 918, and a plurality of energy guides 922, i.e. a first energy guide 922A, a second energy guide 922B, a third energy guide 922C, a fourth energy guide 922D, a fifth energy guide 922E, and a sixth energy guide 922F, which are uniformly separated by about 60 degrees from one another around the circumference of the guidewire lumen 918. As with previous embodiments, it is appreciated that the energy guides 922A-922F need not be uniformly separated from one another around the circumference of the guidewire lumen 918.

Additionally, as with the previous embodiments, each of the energy guides 922A-922F can include a guide distal end (not shown in FIG. 9) that can be positioned at any desired longitudinal position relative to a length of the balloon 904 and/or relative to a length of the guidewire lumen 918. Further, it is also appreciated that the system controller 126 (illustrated in FIG. 1) can control the energy source 124 (illustrated in FIG. 1) and/or the multiplexer 128 (illustrated in FIG. 1) so that light energy is separately directed to each of the energy guides 922A-922F in any desired firing sequence or pattern.

FIG. 10 is a simplified schematic cross-sectional view of still another embodiment of the catheter system 1000. The catheter system 1000 illustrated in FIG. 10 is similar to the catheter systems illustrated and described herein above, except that the catheter system 1000 in FIG. 10 includes a different number of energy guides. More particularly, FIG. 10 illustrates that the catheter system 1000 includes at least a balloon 1004, a catheter shaft 1010, a guidewire lumen 1018, and a plurality of energy guides 1022, i.e. a first energy guide 1022A, a second energy guide 10228, a third energy guide 1022C, a fourth energy guide 1022D, and a fifth energy guide 1022E, which are uniformly separated by about 72 degrees from one another around the circumference of the guidewire lumen 1018. As with previous embodiments, it is appreciated that the energy guides 1022A-1022E need not be uniformly separated from one another around the circumference of the guidewire lumen 1018.

Additionally, as with the previous embodiments, each of the energy guides 1022A-1022E can include a guide distal end (not shown in FIG. 10) that can be positioned at any desired longitudinal position relative to a length of the balloon 1004 and/or relative to a length of the guidewire lumen 1018. Further, it is also appreciated that the system controller 126 (illustrated in FIG. 1) can control the energy source 124 (illustrated in FIG. 1) and/or the multiplexer 128 (illustrated in FIG. 1) so that light energy is separately directed to each of the energy guides 1022A-1022E in any desired firing sequence or pattern.

FIG. 11 is a simplified schematic cross-sectional view of yet another embodiment of the catheter system 1100. The catheter system 1100 illustrated in FIG. 11 is similar to the catheter systems illustrated and described herein above, except that the catheter system 1100 in FIG. 11 includes a different number of energy guides. More particularly, FIG. 11 illustrates that the catheter system 1100 includes at least a balloon 1104, a catheter shaft 1110, a guidewire lumen 1118, and a plurality of energy guides 1122, i.e. a first energy guide 1122A, a second energy guide 1122B, a third energy guide 1122C, and a fourth energy guide 1122D, which are uniformly separated by about 90 degrees from one another around the circumference of the guidewire lumen 1118. As with previous embodiments, it is appreciated that the energy guides 1122A-1122D need not be uniformly separated from one another around the circumference of the guidewire lumen 1118.

Additionally, as with the previous embodiments, each of the energy guides 1122A-1122D can include a guide distal end (not shown in FIG. 11) that can be positioned at any desired longitudinal position relative to a length of the balloon 1104 and/or relative to a length of the guidewire lumen 1118. Further, it is also appreciated that the system controller 126 (illustrated in FIG. 1) can control the energy source 124 (illustrated in FIG. 1) and/or the multiplexer 128 (illustrated in FIG. 1) so that light energy is separately directed to each of the energy guides 1122A-1122D in any desired firing sequence or pattern.

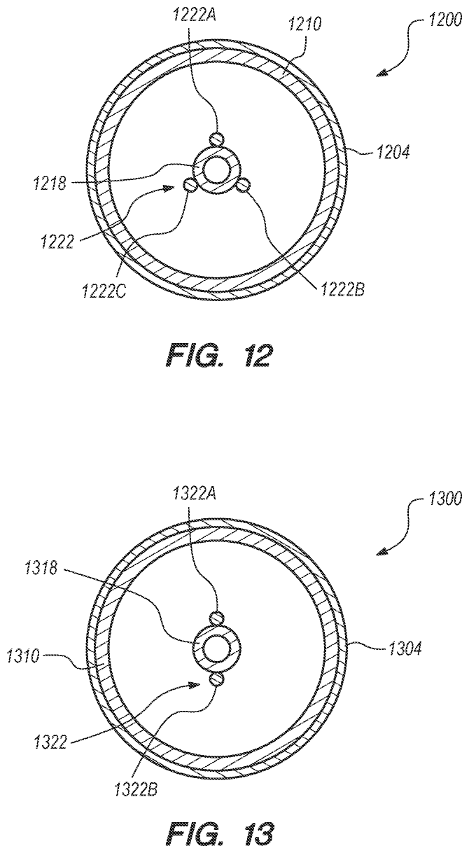

FIG. 12 is a simplified schematic cross-sectional view of still another embodiment of the catheter system 1200. The catheter system 1200 illustrated in FIG. 12 is similar to the catheter systems illustrated and described herein above, except that the catheter system 1200 in FIG. 12 includes a different number of energy guides. More particularly, FIG. 12 illustrates that the catheter system 1200 includes at least a balloon 1204, a catheter shaft 1210, a guidewire lumen 1218, and a plurality of energy guides 1222, i.e. a first energy guide 1222A, a second energy guide 1222B, and a third energy guide 1222C, which are uniformly separated by about 120 degrees from one another around the circumference of the guidewire lumen 1218. As with previous embodiments, it is appreciated that the energy guides 1222A-1222C need not be uniformly separated from one another around the circumference of the guidewire lumen 1218.

Additionally, as with the previous embodiments, each of the energy guides 1222A-1222C can include a guide distal end (not shown in FIG. 12) that can be positioned at any desired longitudinal position relative to a length of the balloon 1204 and/or relative to a length of the guidewire lumen 1218. Further, it is also appreciated that the system controller 126 (illustrated in FIG. 1) can control the energy source 124 (illustrated in FIG. 1) and/or the multiplexer 128 (illustrated in FIG. 1) so that light energy is separately directed to each of the energy guides 1222A-1222C in any desired firing sequence or pattern.

FIG. 13 is a simplified schematic cross-sectional view of still yet another embodiment of the catheter system 1300. The catheter system 1300 illustrated in FIG. 13 is similar to the catheter systems illustrated and described herein above, except that the catheter system 1300 in FIG. 13 includes a different number of energy guides. More particularly, FIG. 13 illustrates that the catheter system 1300 includes at least a balloon 1304, a catheter shaft 1310, a guidewire lumen 1318, and a plurality of energy guides 1322, i.e. a first energy guide 1322A, and a second energy guide 1322B, which are uniformly separated by about 180 degrees from one another around the circumference of the guidewire lumen 1318. As with previous embodiments, it is appreciated that the energy guides 1322A-1322B need not be uniformly separated from one another around the circumference of the guidewire lumen 1318.

Additionally, as with the previous embodiments, each of the energy guides 1322A-1322B can include a guide distal end (not shown in FIG. 13) that can be positioned at any desired longitudinal position relative to a length of the balloon 1304 and/or relative to a length of the guidewire lumen 1318. Further, it is also appreciated that the system controller 126 (illustrated in FIG. 1) can control the energy source 124 (illustrated in FIG. 1) and/or the multiplexer 128 (illustrated in FIG. 1) so that light energy is separately directed to each of the energy guides 1322A-1322B in any desired firing sequence or pattern.

As described in detail herein, in various embodiments, the present invention can be utilized to solve various problems that exist in more traditional catheter systems. For example, by enabling the catheter system to fire each energy guide separately, it is possible to achieve a sequence or firing sequence that could be much more effective at breaking localized lesions. Additionally, firing individual energy guides in a desired firing sequence or pattern can create a moving energy wavefront that more effectively breaks up a lesion at one particular location or an extended lesion. Further, the separate firing of the individual energy guides can be utilized to create a localized shearing force on the leading edge of the spherical wavefront that impinges at an angle (non-normal) relative to the balloon wall, which could have a greater effect in cracking calcified lesions.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

1. A method for treating a vascular lesion within or adjacent to a vessel wall, the method comprising the steps of:
generating light energy with an energy source;
receiving the light energy with a plurality of energy guides; and
controlling the energy source with a system controller of a catheter system so that the light energy from the energy source is sequentially directed to each of the plurality of energy guides in a first firing sequence.

2. The method of claim 1 wherein the step of controlling includes the system controller controlling a firing rate of the energy source to each of the plurality of energy guides.

3. The method of claim 1 wherein the step of controlling includes the system controller controlling the energy source so that light energy from the energy source is alternatively directed to each of the plurality of energy guides at a first firing rate and a second firing rate that is different than the first firing rate.

4. The method of claim 1 wherein the step of receiving includes the plurality of energy guides includes a first energy guide and a second energy guide, and further comprising the steps of positioning a first guide distal end of the first energy guide at a first longitudinal position along a length of the balloon, and positioning a second guide distal end of the second energy guide at a second longitudinal position along the length of the balloon so that the first longitudinal position is different than the second longitudinal position.

5. The method of claim 1 wherein the step of receiving includes the plurality of energy guides includes a first energy guide and a second energy guide, and further comprising the steps of positioning a first guide distal end of the first energy guide at a first longitudinal position along a length of the balloon, and positioning a second guide distal end of the second energy guide at a second longitudinal position along the length of the balloon so that the first longitudinal position is the same as the second longitudinal position.

6. The method of claim 1 further comprising the step of positioning at least a portion of the energy guides within a balloon that is coupled to a catheter shaft.

7. The method of claim 6 wherein the step of controlling includes the system controller controlling a firing sequence to the plurality of energy guides so that an advancing wavefront is generated toward the vascular lesion from near a balloon proximal end and from near a balloon distal end.

8. The method of claim 7 wherein the step of controlling includes the system controller controlling the energy source so that light energy from the energy source is alternatively directed to at least two of the plurality of energy guides at a different firing rate from one another.

9. The method of claim 7 wherein the step of controlling includes the system controller controlling the energy source so that light energy from the energy source is alternatively directed to at least two of the plurality of energy guides at a different firing energy level from one another.

10. The method of claim 9 wherein the firing energy level is dependent at least partially upon the pulse width of the energy pulses.

11. The method of claim 9 wherein the firing energy level is dependent at least partially upon the wavelength of the energy pulses.

12. The method of claim 9 wherein the firing energy level is dependent at least partially upon the amplitude of the energy pulses.

13. The method of claim 6 wherein the step of controlling includes the system controller controlling a firing sequence to the plurality of energy guides so that an advancing wavefront is generated toward the vascular lesion in a direction from one of a balloon proximal end and a balloon distal end.

14. The method of claim 13 wherein the step of controlling includes the system controller controlling the energy source so that light energy from the energy source is alternatively directed to at least two of the plurality of energy guides at a different firing rate from one another.

15. The method of claim 13 wherein the step of controlling includes the system controller controlling the energy source so that light energy from the energy source is alternatively directed to at least two of the plurality of energy guides at a different firing energy level from one another.

16. The method of claim 15 wherein the firing energy level is dependent at least partially upon the pulse width of the energy pulses.

17. The method of claim 15 wherein the firing energy level is dependent at least partially upon the wavelength of the energy pulses.

18. The method of claim 15 wherein the firing energy level is dependent at least partially upon the amplitude of the energy pulses.

19. The method of claim 1 wherein each of the plurality of energy guides includes an optical fiber.

20. The method of claim 1 wherein the energy source is a laser source that generates laser energy.

21. The method of claim 1 wherein the energy source is an energy source that generates electrical impulses.
